SEQUENCE LISTING (21 sequences)

SEQ ID NO: 1 - protein, 22 aa, Artificial Sequence
  VARIANT (1): Xaa is Ala, Val, Phe, Pro, Met, Ile, Leu, Ser, Thr, Tyr, His, Cys, Asn, Gln, Trp or Gly
XXXXXXXXXXXXXXXXXXXXXX

SEQ ID NO: 2 - protein, 78 aa, Artificial Sequence
  VARIANT (1)..(15): Xaa is Gly, Ala, Val, Phe, Pro, Met, Ile, Leu, Asp, Glu, Lys, Arg, Ser, Thr, Tyr, His, Cys, Asn, Gln or Trp
XXXXXXXXXXXXXXXXXXXXXXXXXXXXXXXXXXXXXXXXXXXXXXXXXXXXXXXXXXXXXXXXXXXGXXXXXXGXXX

SEQ ID NO: 3 - protein, 22 aa, Artificial Sequence
  Description of Artificial Sequence amino acid sequence affecting activity of a PME
AVLQNCDIHARKPNSGQKNMVT

SEQ ID NO: 4 - protein, 22 aa, Artificial Sequence
  Description of Artificial Sequence amino acid sequence affecting activity of a PME
AVLQDCDIHARKPNSGQKNMVT

SEQ ID NO: 5 - protein, 22 aa, Artificial Sequence
  Description of Artificial Sequence amino acid sequence affecting activity of a PME
VVFQKCQLVARKPGKYQQNMVT

SEQ ID NO: 6 - protein, 22 aa, Artificial Sequence
  Description of Artificial Sequence amino acid sequence affecting activity of a PME
VVFQKSQLVARKPMSNQKNMVT

SEQ ID NO: 7 - protein, 22 aa, Artificial Sequence
  Description of Artificial Sequence amino acid sequence affecting activity of a PME
GVFQNCKLVCRLPAKGQQCLVT

SEQ ID NO: 8 - protein, 22 aa, Artificial Sequence
  Description of Artificial Sequence amino acid sequence affecting activity of a PME
AVFQNCEFVIRRPMEHQQCIVT

SEQ ID NO: 9 - protein, 22 aa, Artificial Sequence
  Description of Artificial Sequence amino acid sequence affecting activity of a PME
VVFQGCKIMPRQPLSNQFNTIT

SEQ ID NO: 10 - protein, 22 aa, Artificial Sequence
  Description of Artificial Sequence amino acid sequence affecting activity of a PME
FFVQSCKIMPRQPLPNQFNTIT

SEQ ID NO: 11 - protein, 22 aa, Artificial Sequence
  Description of Artificial Sequence amino acid sequence affecting activity of a PME
AVFQNCYLVLRLPRKKGYNVIL

SEQ ID NO: 12 - protein, 22 aa, Artificial Sequence
  Description of Artificial Sequence amino acid sequence affecting activity of a PME
TVIQNSLILCRKGSPGQTNHVT

SEQ ID NO: 13 - DNA, 66 bp, Artificial Sequence
  Description of Artificial Sequence nucleotide sequence capable of coding for amino acid sequence affecting activity of a PME
gccgtgttacaaaattgtgacatccatgcacgaaagcccaattccggccaaaaaaatatggtcaca

SEQ ID NO: 14 - protein, 23 aa, Artificial Sequence
  VARIANT (1): Xaa is Ala, Val, Phe, Pro, Met, Ile, Leu, Ser, Thr, Tyr, His, Cys, Asn, Gln, Trp or Gly
XXWXXXXXXXXXXXXXXXXXXXX

SEQ ID NO: 15 - protein, 9 aa, Artificial Sequence
  Description of Artificial Sequence amino acid sequence affecting activity of a PME
RAWFHECDI

SEQ ID NO: 16 - protein, 9 aa, Artificial Sequence
  Description of Artificial Sequence amino acid sequence affecting activity of a PME
AVWFQNCDI

SEQ ID NO: 17 - protein, 21 aa, Artificial Sequence
  Description of Artificial Sequence amino acid sequence affecting activity of a PME
AVFQNCDIHARKPNDGQKNMV

SEQ ID NO: 18 - protein, 22 aa, Artificial Sequence
  Description of Artificial Sequence amino acid sequence affecting activity of a PME
AVWFQNCDIHARKPNDGQKNMV

SEQ ID NO: 19 - protein, 20 aa, Artificial Sequence
  Description of Artificial Sequence amino acid sequence affecting activity of a PME
AVWFQNCDIRKPNDGQKNMV

SEQ ID NO: 20 - DNA, 13 bp, Artificial Sequence
  Description of Artificial Sequence Low complexity nucleotide sequence
nnnnnnnnnnnnn

SEQ ID NO: 21 - protein, 9 aa, Artificial Sequence
  Description of Artificial Sequence Low complexity protein sequence
XXXXXXXXX

(19) United States
(12) Patent Application Publication
(10) Pub. No.: US 2003/0186417 A1
(43) Pub. Date: Oct. 2, 2003

(54) Amino acid sequence

(75) Inventors: Janne Brunstedt, Roskilde (DK); Tove Martel Ida Else Christensen, Allerod (DK)

Correspondence Address: FOLEY AND LARDNER, SUITE 500, 3000 K STREET NW, WASHINGTON, DC, 20007, US

(73) Assignee: DANISCO A/S

(21) Appl. No.: 10/372,947

(22) Filed: Feb. 26, 2003

Related U.S. Application Data
(63) Continuation of application No. 09/310,113, filed on May 12, 1999.

(30) Foreign Application Priority Data
May 12, 1998 (GB) 9810159.5

(51) Int. Cl. A23L 1/05; A23B 7/10; C07H 21/04; C12P 19/00; C12P 19/04; C12N 9/16

(52) U.S. Cl. 435/196; 435/69.1; 435/320.1; 435/325; 536/23.2; 426/50; 435/101

(57) ABSTRACT

An amino acid sequence is described that affects PME activity. The amino acid has the formula (1):

A1-A2-A3-A4-A5-A6-A7-A8-A9-A10-A11-A12-A13-A14-A15-A16-A17-A18-A19-A20-A21-A22 (I)

The present invention relates to an amino acid sequence. The present invention also relates to a nucleotide sequence coding for same.

In particular, the present invention relates to an amino acid sequence capable of affecting enzymatic activity. The present invention also relates to a nucleotide sequence coding for same.

Pectin is an important commodity in today's industry. For example, it can be used in the food industry as a thickening or gelling agent, such as in the preparation of jams.

Pectin is a structural polysaccharide commonly found in the form of protopectin in plant cell walls. The backbone of pectin comprises α-1,4 linked galacturonic acid residues which are interrupted with a small number of 1,2 linked α-L-rhamnose units. In addition, pectin comprises highly branched regions with an almost alternating rhamno-galacturonan chain. These highly branched regions also contain other sugar units (such as D-galactose, L-arabinose and xylose) attached by glycosidic linkages to the C3 or C4 atoms of the rhamnose units or the C2 or C3 atoms of the galacturonic acid units. The long chains of α-1,4 linked galacturonic acid residues are commonly referred to as “smooth” regions, whereas the highly branched regions are commonly referred to as the “hairy regions”.

Some of the carboxyl groups of the galacturonic residues are esterified (e.g. the carboxyl groups are methylated). Typically esterification of the carboxyl groups occurs after polymerisation of the galacturonic acid residues. However, it is extremely rare for all of the carboxyl groups to be esterified (e.g. methylated). Usually, the degree of esterification will vary from 0-90%. If 50% or more of the carboxyl groups are esterified then the resultant pectin is referred to as a “high ester pectin” (“HE pectin” for short) or a “high methoxyl pectin”. If less than 50% of the carboxyl groups are esterified then the resultant pectin is referred to as a “low ester pectin” (“LE pectin” for short) or a “low methoxyl pectin”. If 50% of the carboxyl groups are esterified then the resultant pectin is referred to as a “medium ester pectin” (“ME pectin” for short) or a “medium methoxyl pectin”. Ifpectinectin does not contain any—or only a few—esterified groups it is usually referred to as pectic acid.

The structure of the pectin, in particular the degree of esterification (e.g. methylation), dictates many of the resultant physical and/or chemical properties of the pectin. For example, pectin gelation depends on the chemical nature of the pectin, especially the degree of esterification. In addition, however, pectin gelation also depends on the soluble-solids content, the pH and calcium ion concentration. With respect to the latter, it is believed that the calcium ions form complexes with free carboxyl groups, particularly those on a LE pectin.

Pectic enzymes are classified according to their mode of attack on the galacturonan part of the pectin molecule. A review of some pectic enzymes has been prepared by Pilnik and Voragen (Food Enzymology, Ed.: P. F.Fox; Elsevier; (1991); pp: 303-337). In particular, pectin methylesterases (EC 3.1.1.11), otherwise referred to as PMEs, de-esterify HE pectins to LE pectins or pectic acids. In contrast, and by way of example, pectin depolymerases split the glycosidic linkages between galacturonosyl methylester residues.

In more detail, PME activity produces free carboxyl groups and free methanol. The increase in free carboxyl groups can be easily monitored by automatic titration. In this regard, earlier studies have shown that some PMEs de-esterify pectins in a random manner, in the sense that they de-esterify any of the esterified (e.g. methylated) galacturonic acid residues on one or more than one of the pectin chains. Examples of PMEs that randomly de-esterify pectins may be obtained from fungal sources such as Aspergillus aculeatus (see WO 94/25575) and Aspergillus japonicus (Ishii et al 1980 J Food Sci 44 pp 611-14). Baron et al (1980 Lebensm. Wiss. M-Technol 13 pp 330-333) apparently have isolated a fungal PME from Aspergillus niger. This fungal PME is reported to have a molecular weight of 39000 D, an isoelectric point of 3.9, an optimum pH of 4.5 and a Kₘ value (mg/ml) of 3.

In contrast, some PMEs are known to de-esterify pectins in a block-wise manner, in the sense that it is believed they attack pectins either at non-reducing ends or next to free carboxyl groups and then proceed along the pectin molecules by a single-chain mechanism, thereby creating blocks of un-esterified galacturonic acid units which can be calcium sensitive. Examples of such enzymes that block-wise enzymatically de-esterify pectin are plant PMEs. Up to 12 isoforms of PME have been suggested to exist in citrus (Pilnik W. and Voragen A. G. J. (Food Enzymology (Ed.: P. F.Fox); Elsevier; (1991); pp: 303-337). These isoforms have different properties.

Random or blockwise distribution of free carboxyl groups can be distinguished by high performance ion exchange chromatography (Schols et al Food Hydrocolloids 19896 pp 115-121). These tests are often used to check for undesirable, residual PME activity in citrus juices after pasteurisation because residual PME can cause, what is called, “cloud loss” in orange juice in addition to a build up of methanol in the juice.

Versteeg et al (J Food Sci 45 (1980) pp 969-971) apparently have isolated a PME from orange. This plant PME is reported to occur in multiple isoforms of differing properties. Isoform I has a molecular weight of 36000 D, an isoelectric point of 10.0, an optimum pH of 7.6 and a Kₘ value (mg/ml) of 0.083. Isoform II has a molecular weight of 36200 D, an isoelectric point of 11.0, an optimum pH of 8.8 and a Kₘ value (mg/ml) of 0.0046. Isoform III (HMW-PE) has a molecular weight of 54000 D, an isoelectric point of 10.2, an optimum pH of 8 and a Kₘ value (mg/ml) of 0.041. However, to date there has been very limited sequence data for such PMEs.

According to Pilnik and Voragen (ibid), PMEs may be found in a number of other higher plants, such as apple, apricot, avocado, banana, berries, lime, grapefruit, mandarin, cherries, currants, grapes, mango, papaya, passion fruit, peach, pear, plums, beans, carrots, cauliflower, cucumber, leek, onions, pea, potato, radish and tomato. However, likewise, to date there has been very limited sequence data for such PMEs.

A plant PME has been reported in WO-A-97/03574. This PME has the following characteristics: a molecular weight of from about 36 kD to about 64 kD; a pH optimum of pH 7-8 when measured with 0.5% lime pectin in 0.15 M NaCl; a temperature optimum of at least 50° C.; a temperature stability in the range of from 10° at least 40° C.; a Kₘ value of 0.07%; an activity maximum at levels of about 0.25 M NaCl; an activity maximum at levels of about 0.2 M Na₂SO₄; and an activity maximum at levels of about 0.3 M NaNO₃.

Another PME has been reported in WO 97/31102.

PMEs have important uses in industry. For example, they can be used in or as sequestering agents for calcium ions. In this regard, and according to Pilnik and Voragen (ibid), cattle feed can be prepared by adding a slurry of calcium hydroxide to citrus peels after juice extraction. After the addition, the high pH and the calcium ions activate any native PME in the peel causing rapid de-esterification of the pectin and calcium pectate coagulation occurs. Bound liquid phase is released and is easily pressed out so that only a fraction of the original water content needs to be removed by expensive thermal drying. The press liquor is then used as animal feed.

As indicated above, a PME has been obtained from Aspergillus aculeatus (WO 94/25575). Apparently, this PME can be used to improve the firmness of a pectin-containing material, or to de-methylate pectin, or to increase the viscosity of a pectin-containing material.

It has also become common to use PME in the preparation of foodstuffs prepared from fruit or vegetable materials containing pectin—such as jams or preservatives. For example, WO-A-94/25575 further reports on the preparation of orange marmalade and tomato paste using PME obtained from Aspergillus aculeatus.

JP-A-63/209553 discloses gels which are obtained by the action of pectin methylesterase, in the presence of a polyvalent metal ion, on a pectic polysaccharide containing as the main component a high-methoxyl poly α-1,4-D-galacturonide chain and a process for their production.

Pilnik and Voragen (ibid) list uses of endogenous PMEs which include their addition to fruit juices to reduce the viscosity of the juice if it contains too much pectin derived from the fruit, their addition as pectinase solutions to the gas bubbles in the albedo of citrus fruit that has been heated to a core temperature of 20° C. to 40° C. in order to facilitate removal of peel and other membrane from intact juice segments (U.S. Pat. No. 4,284,651), and their use in protecting and improving the texture and firmness of several processed fruits and vegetables such as apple (Wiley & Lee 1970 Food Technol 24 1168-70), canned tomatoes (Hsu et al 1965 J Food Sci 30 pp 583-588) and potatoes (Bartolome & Hoff 1972 J Agric Food Chem 20 pp 262-266).

Gahn and Rolin (1994 Food Ingredients Europe, Conf Proceedings pp 252-256) report on the hypothetical application of the industrial “GENU pectin type YM-100” for interacting with sour milk beverages. No details are presented at all on how GENU pectin type YM-100 is prepared.

EP-A-0664300 discloses a chemical fractionation method for preparing calcium sensitive pectin. This calcium sensitive pectin is said to be advantageous for the food industry.

Thus, pectins and de-esterified pectins, in addition to PMEs, have an industrial importance.

We have now found that it is possible to affect the enzymatic activity of a PME by inserting into, deleting from, or converting within a PME, a fairly short amino acid sequence. In this respect, the enzymatic activity of a PME can be altered by inserting or deleting a specific amino acid sequence or converting a sequence to same. However, importantly, the resultant PME is still capable of acting as a PME.

According to the present invention there is provided an amino acid sequence of the formula (I):

A1-A2-A3-A4-A5-A6-A7-A8-A9-A10-A11-A12-A13-A14-A15-A16-A 17-A 18-A19-A20-A211-A22 (1)

wherein

A1 is a hydrophobic or polar amino acid or a neutral amino acid

A2 is a hydrophobic amino acid

A3 is a hydrophobic amino acid

A4 is a polar amino acid

A5 is a polar or charged amino acid or neutral amino acid

A6 is a polar amino acid

A7 is a polar or charged or hydrophobic amino acid

A8 is a hydrophobic amino acid

A9 is a hydrophobic or polar amino acid

A10 is a hydrophobic or polar amino acid

A11 is a charged amino acid

A12 is a charged or polar or hydrophobic amino acid

A13 is a hydrophobic or charged amino acid or neutral amino acid

A14 is a hydrophobic or polar amino acid or charged or neutral amino acid

A15 is a charged or polar or hydrophobic amino acid

A16 is a polar or hydrophobic or charged amino acid or neutral amino acid

A17 is a polar or charged amino acid or neutral amino acid

A18 is a polar or charged or hydrophobic amino acid

A19 is a polar amino acid or a neutral amino acid

A20 is a hydrophobic or polar amino acid

A21 is a hydrophobic amino acid

A22 is a polar or hydrophobic amino acid.

As indicated, we have found that the amino acid sequence of formula (I) affects PME activity.

In particular, we have found that the amino acid sequence of formula (I) plays a role in whether a PME is capable of block-wise de-esterifying a PME substrate or randomly de-esterifying a PME substrate.

More in particular, we have found that the presence of the amino acid sequence of formula (I) in a PME means that the PME is capable of block-wise de-esterifying a PME substrate. On the other hand, the absence of some or all of the amino acid sequence of formula (I) in a PME means that the PME is capable of randomly de-esterifying a PME substrate.

These results are surprising—in the sense that a relatively short amino acid sequence can govern to at least some extent the type of activity of an enzyme, especially a PME.

The present invention also covers the use of an amino acid sequence of formula (I) for affecting enzymatic activity.

The present invention also covers a modified enzyme comprising the amino acid sequence of the formula (I).

The present invention also covers a foodstuff prepared by use of the amino acid sequence of the formula (I).

Preferably the foodstuff is a pectin.

The present invention also covers a modified PME comprising the amino acid sequence of formula (I).

The present invention also covers a process of de-methylating pectin comprising contacting pectin with a modified PME comprising the amino acid sequence of formula (I).

The present invention also covers a process of preparing a foodstuff comprising using a de-methylated pectin, wherein the de-methylated pectin is prepared by contacting pectin with a modified PME comprising the amino acid sequence of formula (I).

In particular, the present invention also covers a PME enzyme comprising the amino acid sequence of formula (I). However, in this embodiment, the present invention does not cover a native PME when it is in its natural environment and when it has been expressed by its native nucleotide coding sequence which is also in its natural environment and when that nucleotide sequence is under the control of its native promoter which is also in its natural environment. For simplicity, this embodiment of the present invention is called “a non-native PME”.

The present invention also encompasses nucleotide sequences coding for the amino acid sequence of formula (I).

Thus, the present invention also covers a nucleotide sequence coding for a PME enzyme comprising the amino acid sequence of formula (I). However, in this embodiment, the present invention does not cover a native PME coding gene when it is in its natural environment and when that gene is under the control of its native promoter which is also in its natural environment. For simplicity, this embodiment of the present invention is called “a non-native PME coding gene”.

The present invention also encompasses constructs, vectors, plasmids, cells, tissues, organs and organisms comprising or capable of expressing the amino acid sequence of formula (I)—including it being part of a larger amino acid sequence (e.g. as a PME enzyme)—and/or the nucleotide sequence of the present invention.

Other aspects of the present invention include methods of expressing or allowing expression or transforming any one of the nucleotide sequence, the construct, the plasmid, the vector, the cell, the tissue, the organ or the organism, as well as the products thereof.

The present invention also encompasses amino acid sequences that are at least 80% homologous with the amino acid sequence of formula (I), preferably amino acid sequences that are at least 85% homologous with the amino acid sequence of formula (I), preferably amino acid sequences that are at least 90% homologous with the amino acid sequence of formula (I), preferably amino acid sequences that are at least 95% homologous with the amino acid sequence of formula (I), preferably amino acid sequences that are at least 98% homologous with the amino acid sequence of formula (I). In a highly preferred embodiment, the amino acid sequence is the same as the amino acid sequence of formula (I).

In particular, the term “homology” as used herein may be equated with the term “identity”. Here, sequence homology with respect to the nucleotide sequence of the present invention can be determined by a simple “eyeball” comparison (i.e. a strict comparison) of any one or more of the sequences with another sequence to see if that other sequence has at least 75% identity to the sequence(s). Relative sequence homology (i.e. sequence identity) can also be determined by commercially available computer programs that can calculate % homology between two or more sequences. A typical example of such a computer program is CLUSTAL.

The present invention also encompasses nucleotide sequences that code for an amino acid sequence that are at least 80% homologous with the amino acid sequence of formula (I), preferably amino acid sequences that are at least 85% homologous with the amino acid sequence of formula (I), preferably amino acid sequences that are at least 90% homologous with the amino acid sequence of formula (I), preferably amino acid sequences that are at least 95% homologous with the amino acid sequence of formula (I), preferably amino acid sequences that are at least 98% homologous with the amino acid sequence of formula (I). In a highly preferred embodiment, the amino acid sequence is the same as the amino acid sequence of formula (I).

Likewise, here the term “homology” as used herein may be equated with the term “identity”. Here, sequence homology with respect to the nucleotide sequence of the present invention can be determined by a simple “eyeball” comparison (i.e. a strict comparison) of any one or more of the sequences with another sequence to see if that other sequence has at least 75% identity to the sequence(s). Relative sequence homology (i.e. sequence identity) can also be determined by commercially available computer programs that can calculate % homology between two or more sequences. A typical example of such a computer program is CLUSTAL.

The term “vector” includes expression vectors and transformation vectors.

The term “expression vector” means a construct capable of in vivo or in vitro expression.

The term “transformation vector” means a construct capable of being transferred from one species to another—such as from an E. coli to a filamentous fungus (e.g. Aspergillus) or to a non-filamentous fungus (e.g. Pichia). It may even be a construct capable of being transferred from an E. coli to an Agrobacterium to a plant.

The term “tissue” includes isolated tissue and tissue within an organ. The tissue may be a plant tissue.

The term “organism” in relation to the present invention includes any organism (including micro-organisms and unicellular organisms) that could comprise the nucleotide sequence according to the present invention and/or products obtained therefrom, wherein the nucleotide sequence according to the present invention can be expressed when present in the organism. A preferred organism is a micro-organism—such as a fungus—such as Aspergillus or yeast. The organism may also be a plant.

The transformed cell or organism could prepare acceptable quantities of the desired PME which would be easily retrievable from, the cell or organism.

Preferably the construct of the present invention comprises the nucleotide sequence of the present invention and a promoter.

The term “promoter” is used in the normal sense of the art, e.g. an RNA polymerase binding site in the Jacob-Monod theory of gene expression.

In one aspect, the promoter of the present invention is capable of expressing the nucleotide sequence of the present invention.

In one aspect, the nucleotide sequence according to the present invention (such as that coding for a PME according to the present invention) may be under the control of a promoter that may be a cell or tissue specific promoter. If, for example, the organism is a plant then the promoter can be one that affects expression of the nucleotide sequence in any one or more of fruit, seed, stem, sprout, root and leaf tissues. The promoter may additionally include features to ensure or to increase expression in a suitable host. For example, the features can be conserved regions such as a Pribnow Box or a TATA box.

The construct of the present invention may even contain other sequences to affect (such as to maintain, enhance, decrease) the levels of expression of the nucleotide sequence of the present invention. For example, suitable other sequences include the Shl-intron or an ADH intron. Other sequences include inducible elements—such as temperature, chemical, light or stress inducible elements. Also, suitable elements to enhance transcription or translation may be present. An example of the latter element is the TMV 5′ signal sequence (see Sleat Gene 217 [1987] 217-225; and Dawson Plant Mol. Biol. 23 [1993] 97).

The present invention also encompasses combinations of promoters and/or nucleotide sequences coding for proteins or recombinant enzymes and/or elements.

The amino-acid sequence of formula (I) may even be used to screen for PME enzymes that may be capable of exhibiting block-wise de-esterification of a PME substrate. For example, the screening may be performed on a computer database. Alternatively, or in addition, the amino-acid sequence of formula (I) may be used to generate anti-bodies that are capable eliciting a detectable immune response/reaction with sequences that are the same as the amino-acid sequence of formula (I). These anti-bodies may then be used to screen for PME enzymes that may be capable of exhibiting block-wise de-esterification of a PME substrate.

Thus, the present invention also covers the use of the amino-acid sequence of formula (I) or an anti-body thereto to screen for a PME enzyme that may be capable of exhibiting block-wise de-esterification of a PME substrate.

Antibodies can be raised against the enzyme of the present invention by injecting rabbits with the purified enzyme and isolating the immunoglobulins from antiserum according to procedures described according to N Harboe and A Ingild (“Immunization, Isolation of Immunoglobulins, Estimation of Antibody Titre” In A Manual of Quantitative Immunoelectrophoresis, Methods and Applications, N H Axelsen, et al (eds.), Universitetsforlaget, Oslo, 1973) and by T G Cooper (“The Tools of Biochemistry”, John Wiley & Sons, New York, 1977). By way of example, the amino acid sequence of formula (I) can be cross linked to a dipthteria toxoid carrier. Antibodies are then raised against the conjugate. Screening for PMEs comprising the amino acid sequence of formula (I) can then be carried out using inter alia the anti-bodies and SDS-PAGE (see Marcussen and Poulsen 1991 Analytical Biochem 198: 318-323).

The present invention also covers a PME enzyme identified by such a screen.

The nucleotide sequence coding for the amino-acid sequence of formula (I)—or even a sequence capable of hybridising therewith (preferably under stringent conditions—e.g. 65° C. and 0.1 SSC {1×SSC=0.15 M NaCl, 0.015 Na₃ citrate pH 7.0}) may also be used to screen for genes coding for PME enzymes that may be capable of exhibiting block-wise de-esterification of a PME substrate. For example, the screening may be performed on a library of clones or even on a computer database.

Thus, the present invention also covers the use of the nucleotide sequence coding for the amino-acid sequence of formula (I) or a sequence that is capable of hybridising therewith to screen for a gene coding a PME enzyme that may be capable of exhibiting block-wise de-esterification of a PME substrate.

The present invention also covers a gene coding for a PME enzyme identified by such a screen.

The nucleotide sequence of the present invention may also be used to devise antisense sequences that may be capable of silencing the PME coding gene that includes a region coding for the amino acid sequence of formula (I). Thus, the antisense nucleotide sequences may be able to selectively switch off a PME.

The present invention is advantageous in that it provides a means to affect PME activity by use of a relatively short amino acid sequence and/or a nucleotide sequence coding for same.

The amino acid sequence of formula (I) can be introduced into an existing PME by use of appropriate chemical or biological techniques. Wherever appropriate, the amino acid sequence of formula (I) may be introduced in part, in whole or even as part of larger fragment. Preferably, the resultant amino acid sequence of formula (I) is positioned near to the C terminal part of the PME active site.

In this respect, we believe that the PME active site (which may be called the catalytic site) may be typically characterised by the amino acid of sequence of formula (II):

| | |
|---|---|
| N-N-N-N-N-N-N-N-N-N-N-N-N-N-N-H-H-N-H-N-N-N-N-N-N-N-N-N-N-N-N-N-H-N- | (II) |
| N-N-P-C-P-H-N-H-N-N-N-N-N-N-N-N-N-N-N-N-N-N-N-N-N-N-N-N-N-N-N-H-N-G- | |
| N-N-C-N-H-H-G-N-N-N | |

wherein

H independently represents a hydrophobic amino acid

C independently represents a charged amino acid

P independently represents a polar amino acid

G represents glycine

N independently represents glycine or a hydrophobic or charged or polar amino acid.

For the amino acid sequence of formula (II): examples of hydrophobic amino acids include: Ala (A), Val (V), Phe (F), Pro (P), Met (M), Ile (I), Leu (L); examples of charged amino acids include Asp (D), Glu (E), Lys (K), Arg (R); and examples of polar amino acids include: Ser (S), Thr (T), Tyr (Y), His (H), Cys (C), Asn (N), Gln (O), Trp (W).

We believe that the amino acid sequence of formula (II) is important for defining the active site since previous studies have shown that for Aspergillus PME amino acid 14 is involved in the active site as changing a histidine into an alanine caused a loss of PME activity (Duwe and Khanh (1996) Biotechn Letters vol 18: 621-626).

Alternatively, it may be possible to alter an existing amino acid sequence contained within a PME by, for example, making one or more amino acids more polar by use of site specific chemical alterations and, in doing so, convert that amino acid sequence to a sequence having the formula (I) and thus converting the PME to a PME that can block-wise de-esterify a PME substrate.

Alternatively, the coding sequence for a PME may be altered by insertion or deletion or substitution of a nucleotide sequence coding for the amino acid sequence of formula (I). Wherever appropriate, the nucleotide sequence coding for an amino acid sequence of formula (I) may be introduced in part, in whole or even as part of larger fragment. Insertion by means of a larger fragment may be appropriate, for example, when two suitable restriction sites are not in the exact required location. In this respect, it may be necessary to remove a larger fragment from the initial gene and then replace it with a second fragment that comprises the nucleotide sequence coding for the amino acid sequence of formula (I) and wherein that nucleotide sequence may be flanked one or both sides by a sequence at least substantially similar to at least a part of the nucleotide sequence fragment that has been removed. Preferably, the resultant nucleotide sequence coding for the amino acid sequence of formula (I) is positioned near to the 3′ end of the PME active site.

In this respect, if it is desired to adapt a PME enzyme that normally exhibits block-wise desterification properties, then it is possible to remove or substitute one or more of the codons of the nucleotide coding sequence coding for the amino acid sequence of formula (I)—or even add one or more additional codons to that nucleotide sequence—and in doing so convert the nucleotide sequence from being one that does code for an amino acid sequence of formula (I) to one that does not code for an amino acid sequence of formula (I) and thus change the activity of the PME so that it is capable of exhibiting random desterification properties.

In this respect, if it is desired to adapt a PME enzyme that normally exhibits random desterification properties, then it is possible to remove or substitute one or more of the codons of a nucleotide coding sequence contained within the PME coding sequence (but not the active site thereof)—or even add one or more additional codons to that nucleotide sequence—and in doing so convert a part of the nucleotide sequence from being one that does not code for an amino acid sequence of formula (I) to one that does code for an amino acid sequence of formula (I) and thus change the activity of the PME so that it is capable of exhibiting block-wise desterification properties.

By way of example, it is possible to splice out a section of a PME coding gene from, for example, Aspergillus and then replace that section with a nucleotide sequence coding for an amino acid sequence of formula (I). The resultant modified Aspergillus PME will then be capable of exhibiting block-wise de-esterification properties on PME substrates.

Thus, the present invention encompasses a modified PME wherein the modified PME is obtainable from providing an initial PME that does not comprise an amino acid sequence of the formula (I); and modifying the initial PME so that it does comprise an amino acid sequence of the formula (I).

The present invention also encompasses a modified PME wherein the modified PME is obtainable from providing an initial PME that does comprise an amino acid sequence of the formula (I); and modifying the initial PME so that it does not comprise an amino acid sequence of the formula (I).

The present invention also encompasses a modified PME wherein the modified PME is obtained from providing an initial PME that does not comprise an amino acid sequence of the formula (I); and modifying the initial PME so that it does comprise an amino acid sequence of the formula (I).

The present invention also encompasses a modified PME wherein the modified PME is obtained from providing an initial PME that does comprise an amino acid sequence of the formula (I); and modifying the initial PME so that it does not comprise an amino acid sequence of the formula (I).

In addition, the present invention encompasses a process of modifying a PME comprising the steps of providing an initial PME that does not comprise an amino acid sequence of the formula (I); and modifying the initial PME so that it does comprise an amino acid sequence of the formula (I).

The present invention also encompasses a process of modifying a PME comprising the steps of providing an initial PME that does comprise an amino acid sequence of the formula (I); and modifying the initial PME so that it does not comprise an amino acid sequence of the formula (I).

The present invention also encompasses a modified PME wherein the modified PME is obtainable from providing an initial PME that comprises an initial amino acid sequence of the formula (I); and modifying the initial PME so that it comprises a modified amino acid sequence of the formula (I), wherein the initial amino acid sequence of the formula (I) is different to the modified amino acid sequence of the formula (I).

The present invention also encompasses a modified PME wherein the modified PME is obtained from providing an initial PME that comprises an initial amino acid sequence of the formula (I); and modifying the initial PME so that it comprises a modified amino acid sequence of the formula (I), wherein the initial amino acid sequence of the formula (I) is different to the modified amino acid sequence of the formula (I).

In addition, the present invention encompasses a process of modifying a PME comprising the steps of providing an initial PME that comprises an initial amino acid sequence of the formula (I); and modifying the initial PME so that it comprises a modified amino acid sequence of the formula (I), wherein the initial amino acid sequence of the formula (I) is different to the modified amino acid sequence of the formula (I).

These last three aspects may be of importance should it be desirable to introduce a different block-wise de-esterification activity.

In accordance with the present invention it is possible to insert all or part (such as one or more amino acid sequences of the formula (I)) of the amino acid sequence of the formula (I) into a PME such that the resultant modified PME comprises all of the amino acid sequence of the formula (I). The modification aspect of the present invention also includes modifying existing amino acid residues in a PME such that the resultant PME comprises the amino acid sequence of the formula (I).

As indicated, the modification step can include any one or more of addition, substitution or deletion of one or more amino acids.

In order to ensure the correct folding pattern of the modified enzyme it may be necessary to remove one or more amino acid residues. If it is necessary to remove one or more amino acid residues then usually those residue(s) are removed at the point of insertion of all or part of the amino acid sequence of formula (I). By way of example, if the full length amino acid sequence of formula (I) is inserted into a sequence to form a modified enzyme then it may be necessary to remove a 22 amino acid portion from the enzyme. Naturally, the removal step can take place before, during or after the insertion step.

The present invention also encompasses a gene coding for a modified PME wherein the gene coding for the modified PME is obtainable from providing an initial gene coding for a PME that does not comprise a sequence coding for an amino acid sequence of the formula (I); and modifying the initial gene coding for the PME so that it does comprise a nucleotide sequence coding for an amino acid sequence of the formula (I).

The present invention also encompasses a gene coding for a modified PME wherein the gene coding for the modified PME is obtainable from providing an initial gene coding for a PME that does comprise a sequence coding for an amino acid sequence of the formula (I); and modifying the initial gene coding for the PME so that it does not comprise a nucleotide sequence coding for an amino acid sequence of the formula (I).

The present invention also encompasses a gene coding for a modified PME wherein the gene coding for the modified PME is obtained from providing an initial gene coding for a PME that does not comprise a sequence coding for an amino acid sequence of the formula (I); and modifying the initial gene coding for the PME so that it does comprise a nucleotide sequence coding for an amino acid sequence of the formula (I).

The present invention also encompasses a gene coding for a modified PME wherein the gene coding for the modified PME is obtained from providing an initial gene coding for a PME that does comprise a sequence coding for an amino acid sequence of the formula (I); and modifying the initial gene coding for the PME so that it does not comprise a nucleotide sequence coding for an amino acid sequence of the formula (I).

The present invention also encompasses a method of preparing a gene coding for a modified PME comprising the steps of providing an initial gene coding for a PME that does not comprise a sequence coding for an amino acid sequence of the formula (I); and modifying the initial gene coding for the PME so that it does comprise a nucleotide sequence coding for an amino acid sequence of the formula (I).

The present invention also encompasses a method of preparing a gene coding for a modified PME comprising the steps of providing an initial gene coding for a PME that does comprise a sequence coding for an amino acid sequence of the formula (I); and modifying the initial gene coding for the PME so that it does not comprise a nucleotide sequence coding for an amino acid sequence of the formula (I).

The present invention also encompasses a gene coding for a modified PME wherein the gene coding for the modified PME is obtainable from providing an initial gene coding for a PME that comprises a sequence coding for an initial amino acid sequence of the formula (I); and modifying the initial gene coding for the PME so that it comprises a nucleotide sequence coding for a modified amino acid sequence of the formula (I), and wherein the initial amino acid sequence of the formula (I) is different to the modified amino acid sequence of the formula (I).

The present invention also encompasses a gene coding for a modified PME wherein the gene coding for the modified PME is obtained from providing an initial gene coding for a PME that comprises a sequence coding for an initial amino acid sequence of the formula (I); and modifying the initial gene coding for the PME so that it comprises a nucleotide sequence coding for a modified amino acid sequence of the formula (I), and wherein the initial amino acid sequence of the formula (I) is different to the modified amino acid sequence of the formula (I).

The present invention also encompasses a method for preparing a gene coding for a modified PME comprising the steps of providing an initial gene coding for a PME that comprises a sequence coding for an initial amino acid sequence of the formula (I); and modifying the initial gene coding for the PME so that it comprises a nucleotide sequence coding for a modified amino acid sequence of the formula (I), and wherein the initial amino acid sequence of the formula (I) is different to the modified amino acid sequence of the formula (I).

These last three aspects may be of importance should it be desirable to introduce a different block-wise de-esterifaction activity.

In accordance with the present invention it is also possible to insert all or part (such as one or more nucleotide sequences coding for the amino acid sequence of the formula (I)) of a nucleotide sequence coding for the amino acid sequence of the formula (I) into a gene coding for a PME such that the resultant gene codes for a modified PME comprising all of the amino acid sequence of the formula (I).

As indicated, the modification step can include any one or more of addition, substitution or deletion of one or more nucleotides.

In order to ensure the correct folding pattern of the resultant expressed modified enzyme it may be necessary to remove one or more nucleotides. If it is necessary to remove one or more nucleotides then usually those nucleotide(s) are removed at the point of insertion of all or part of the nucleotide sequence coding for the amino acid sequence of formula (I). By way of example, if the full length nucleotide sequence coding for the amino acid sequence of formula (I) is inserted into a sequence to form a modified enzyme then it may be necessary to remove a 66 nucleotide portion from the enzyme coding sequence. Naturally, the removal step can take place before, during or after the insertion step.

The PME of the present invention may be obtained from modifying a PME from natural sources or even obtained from natural sources or it may be chemically synthesised. For example, the PME for modification may be obtainable from a fungus, such as by way of example a PME of fungal origin (i.e. a PME that has been obtained from a fungus). Alternatively, the PME for modification may be obtainable from a bacterium, such as by way of example a PME of bacterial origin (i.e. a PME that has been obtained from a bacterium). Alternatively, the PME for modification may be obtainable from a plant, such as by way of example a PME of plant origin (i.e. a PME that has been obtained from a plant). In one preferred embodiment, the PME of the present invention is prepared by use of recombinant DNA techniques.

Likewise, the gene coding for the PME of the present invention may be obtained from modifying a gene coding for a PME from natural sources or even obtained from natural sources or it may be chemically synthesised. For example, the gene coding for a PME for modification may be obtainable from a fungus, such as by way of example a gene coding for a PME of fungal origin (i.e. a gene coding for a PME that has been obtained from a fungus). Alternatively, the gene coding for a PME for modification may be obtainable from a bacterium, such as by way of example a gene coding for a PME of bacterial origin (i.e. a gene coding for a PME that has been obtained from a bacterium). Alternatively, the gene coding for a PME for modification may be obtainable from a plant, such as by way of example a gene coding for a PME of plant origin (i.e. a gene coding for a PME that has been obtained from a plant). In one preferred embodiment, the gene coding for a PME of the present invention is prepared by use of recombinant DNA techniques.

Thus, a key element of the present invention relates to the amino acid sequence of the formula (I) as well as a nucleotide sequence coding for same.

Preferably, A1 is a hydrophobic amino acid.

Preferably A5 is a polar amino acid.

Preferably A7 is a polar amino acid.

Preferably A9 is a hydrophobic amino acid.

Preferably A10 is a hydrophobic amino acid.

Preferably A12 is a charged amino acid.

Preferably A13 is a hydrophobic amino acid.

Preferably A14 is a hydrophobic amino acid.

Preferably A15 is a charged amino acid.

Preferably A16 is a polar amino acid.

Preferably A17 is a polar amino acid.

Preferably A18 is a polar amino acid.

Preferably A20 is a hydrophobic amino acid.

Preferably A22 is a polar amino acid.

As indicated, the amino acid sequence of formula (I) comprises a grouping of one or more hydrophobic amino acids, polar amino acids, charged amino acids and neutral amino acids. Any one or more of the hydrophobic amino acids, polar amino acids, charged amino acids or neutral amino acids can be a non-natural amino acid. In this respect, it may be possible—for example—to derivatise a non-polar naturally occurring amino acid so that it becomes a polar amino acid. Teachings on non-natural amino acids can be found in Creighton (1984 Proteins: Structures and Molecula Principles. W. H. Freeman and Company, New York, USA). This reference also provides some general teachings on the modification of amino acid residues—such as glycosylation, phosphorylation and acetylation.

In one preferred aspect, however, the hydrophobic amino acids, polar amino acids, charged amino acids, neutral amino acids are naturally occurring amino acids.

For the amino acid sequence of formula (I), preferable examples of hydrophobic amino acids include: Ala (A), Val (V), Phe (F), Pro (P), Met (M), Ile (I), Leu (L).

For the amino acid sequence of formula (I), preferable examples of charged amino acids include Asp (D), Glu (E), Lys (K), Arg (R).

For the amino acid sequence of formula (I), preferable examples of polar amino acids include: Ser (S), Thr (T), Tyr (Y), His (H), Cys (C), Asn (N), Gin (O), Trp (W).

For the amino acid sequence of formula (I), a preferable example of a neutral amino acid is glycine (G).

Preferably A1 is A, V, G or T.

Preferably A2 is V or L.

Preferably A3 is L, F or I.

Preferably A4 is Q.

Preferably A5 is N, D, K, G or S.

Preferably A6 is C or S.

Preferably A7 is D, Q, K, E, Y or L.

Preferably A8 is I, L or F.

Preferably A9 is H, N, V, M or L.

Preferably A10 is A, C, I, P, L, C or S.

Preferably A11 is R.

Preferably A12 is K, R, L, Q or Y.

Preferably A13 is P, G or R.

Preferably A14 is N, G, M, A, L, R or S.

Preferably A15 is S, K, E, P or D.

Preferably A16 G, Y, H, N, K or V.

Preferably A17 is Q, G or K.

Preferably A18 is K, Q, F, Y, T or S.

Preferably A19 is N, C or G.

Preferably A20 is M, L, I, T, V, H or N.

Preferably A21 is V or I.

Preferably A22 is T, L or S.

Once the modified PME has been prepared in accordance with the present invention or quantities of PME that has been identified using the screen of the present invention have been prepared, then that PME of the present invention may be added to one or more PME substrate(s). The PME substrates may be obtainable from different sources and/or may be of different chemical composition.

In a preferred embodiment, at least one of the PME substrates is pectin or is a substrate that is derivable from or derived from pectin (eg. a pectin derivative).

The term “derived from pectin” includes derivatised pectin, degraded (such as partially degraded) pectin and modified pectin. An example of a modified pectin is pectin that has been prior treated with an enzyme such as a PME. An example of a pectin derivative is pectin that has been chemically treated—eg. amidated.

In addition, the PME of the present invention can be used in conjunction with additional, and optionally different, PME(s).

If there is more than one PME present, then the PMEs may be obtainable from different sources and/or may be of different composition and/or may have a different reactivity profile (e.g. different pH optimum and/or different temperature optimum).

With the present invention, the PME enzyme of the present invention may de-esterify the PME substrates in a random manner or in a block-wise manner. If there is more than one PME, then each PME is independently selected from a PME that can de-esterify the PME substrate(s) in a random manner or a PME that can de-esterify the PME substrate(s) in a block-wise manner.

In one preferred embodiment, the (or at least one) modified PME enzyme of the present invention de-esterifies the PME substrate(s) in a block-wise manner.

In a further preferred embodiment, the modified PME enzyme of the present invention has a low pH optimum (such as from pH 2 to 5, preferably from pH 2.5 to 4.5) and a high affinity for pectin (such as<1 mg/ml) and the ability to de-methylate pectin in a block-wise manner.

If there is more than one PME, then each PME is independently selected from a PME enzyme that is sensitive to sodium ions (Na-sensitive) or a PME enzyme that is insensitive to sodium ions (Na-insensitive). In one preferred embodiment, the (or at least one) PME enzyme is a PME enzyme that is Na-sensitive.

The additional PME may be obtainable from natural sources or even obtained from natural sources or it may be chemically synthesised. For example, the additional PME may be obtainable from a fungus, such as by way of example a PME of fungal origin (i.e. a PME that has been obtained from a fungus). Alternatively, the additional PME may be obtainable from a bacterium, such as by way of example a PME of bacterial origin (i.e. a PME that has been obtained from a bacterium). Alternatively, the additional PME may be obtainable from a plant, such as by way of example a PME of plant origin (i.e. a PME that has been obtained from a plant). In one preferred embodiment, the additional PME is prepared by use of recombinant DNA techniques. For example, the additional PME can be a recombinant PME as disclosed in WO-A-97/03574 or the PME disclosed in either WO-A-94/25575 or WO-A-97/31102 as well as variants, derivatives or homologues of the sequences disclosed in those patent applications. In one preferred embodiment the additional PME is the recombinant PME of WO-A-97/03574 (the contents of which are incorporated herein by reference) and/or the PME of WO-A-94/25575 (the contents of which are incorporated herein by reference), or a variant, derivative or homologue thereof.

It is believed that pectin de-esterified by the modified PME may have a different structure than that de-esterified by the non-modified PME. In this respect, if the non-modified PME does not comprise the amino acid sequence of formula (I) whereas the modified PME does then the pectin treated by the modified PME may be at least partially de-esterified in a blockwise manner—is opposed to a random manner with the non-modified PME. In addition, it is believed that aspects such as calcium sensitivity of the PME treated pectin may also change depending on whether or not the modified PME comprises the amino acid sequence of formula (I). It is believed that if the modified PME does comprise the amino acid sequence of formula (I) then the PME treated pectin may have a higher calcium sensitivity than the pectin treated by the unmodified PME.

It is also believed that the overall affinity of the PME for pectin may change upon modification.

It is also believed that there may be a change in the pH optimum for the PME upon modification.

This means that it may be possible to tailor a modified PME to suit individual requirements—such as optimal reaction conditions. Thus, it may be possible to modify a plant PME that has a high pH optimum and the ability to de-esterify pectin in a block-wise manner to a modified PME that still has a high pH optimum but wherein the PME now has the ability to de-esterify pectin in a random manner simply by removing, altering or silencing (such as by selective antisense technology) the amino acid sequence of formula (I) or the sequence coding for same. Likewise, it may be possible to modify a fungal PME or a bacterial PME that has a low pH optimum and the ability to de-esterify pectin in a random manner to a modified PME that still has a low pH optimum but wherein the PME now has the ability to de-esterify pectin in at least a partial block-wise manner simply by introducing an amino acid sequence of formula (I) or converting an existing section of the sequence to same and/or altering the coding sequence to code for same.

The PME of the present invention can be used to prepare a foodstuff.

The term “foodstuff” can include food for human and/or animal consumption. Typical foodstuffs include jams, marmalades, jellies, dairy products (such as milk or cheese), meat products, poultry products, fish products and bakery products. The foodstuff may even be a beverage. The beverage can be a drinking yoghurt, a fruit juice or a beverage comprising whey protein.

The PME of the present invention may be used in conjunction with other types of enzymes.

Examples of other types of enzymes include other pectinases, pectin depolymerases, poly-galacturonases, pectate lyases, pectin lyases, rhamno-galacturonases, galactanases, cellulases, hemicellulases, endo-β-glucanases, arabinases, acetyl esterases, or pectin releasing enzymes, or combinations thereof.

Examples of amino-acid sequences of the formula (I) include:

AVLQNCDIHARKPNSGQKNMVT
AVLQDCDINARRPNSGQKNMVT
VVFQKCQLVARKPGKYQQNMVT
VVFQKCQLVARKPGKYQQNMVT
VVFQKSQLVARKPMSNQKNMVT
GVFQNCKLVCRLPAKGQQCLVT
AVFQNCEFVIRRPMEHQQCIVT
VVFQGCKIMPRQPLSNQFNTIT
FFVQSCKIMPRQPLPNQFNTIT
AVFQNCYLVLRLPRKKGYNVIL
TVIQNSLILCRKGSPGQTNHVT

As indicated above, the present invention encompasses nucleotide sequences coding for the amino acid sequence of formula (I). Naturally, the skilled person can select the approprate collection of codons that would ultimately yield a nucleotide sequence capable of coding for an anmino acid sequence of the formula (I). By way of a non-limiting example, an example of a suitable amino acid sequence of the formula (I) would be:

AVLQNCDIHARKPNSGQKNMVT

and a suitable nucleotide coding sequence would be:

GCCGTGTTACAAAATTGTGACATCCATGCACGAAAGCCCAATTCCGGCCA
AAAAAATATGGTCACA.

In accordance with the present invention it is possible to prepare transformed cells, transformed organs or transformed organisms wherein endogenous PME production has been halted or suppressed or removed and wherein exogenous modified PME according to the present invention is expressed instead. The cell may be a plant cell. The organ may be a plant organ. Preferably the organism is a fungus (such as Aspergillus or yeast). The organism may even be a plant. This aspect of the present invention has the advantage in that, for example, transformed plants according to the present invention, on ripening will produce one or more different types of pectins than would the non-modified plant cells.

Even though WO-A-97/03574 does not even suggest the PME of the present invention, let alone the amino acid sequence of formula (I), its teachings do provide some useful teachings on how to prepare a PME according to the present invention by use of a modified gene coding for PME (such as by way of one of the modifications outlined above). In addition, these teachings also provide a good background on how to prepare transformed cells, transformed organs, and transformed organisms that are capable of expressing the amino acid sequence of the formula (I) alone and when part of a larger component (such as when part of a PME according to the present invention). Some of these teachings are recited below.

In order to express a recombinant PME, the host organism can be a prokaryotic or a eukaryotic organism. Examples of suitable prokaryotic hosts include E. coli and Bacillus subtilis. Teachings on the transformation of prokaryotic hosts is well documented in the art, for example see Sambrook et al (Molecular Cloning: A Laboratory Manual, 2nd edition, 1989, Cold Spring Harbor Laboratory Press). If a prokaryotic host is used then the gene may need to be suitably modified before transformation—such as by removal of introns.

In one embodiment, the host organism can be of the genus Aspergillus, such as Aspergillus niger. A transgenic Aspergillus can be prepared by following the teachings of Rambosek, J. and Leach, J. 1987 (Recombinant DNA in filamentous fungi: Progress and Prospects. CRC Crit. Rev. Biotechnol. 6:357-393), Davis R. W. 1994 (Heterologous gene expression and protein secretion in Aspergillus. In: Martinelli S. D., Kinghorn J. R. (Editors) Aspergillus: 50 years on. Progress in industrial microbiology vol 29. Elsevier Amsterdam 1994. pp 525-560), Ballance, D. J. 1991 (Transformation systems for Filamentous Fungi and an Overview of Fungal Gene structure. In: Leong, S. A., Berka R.M. (Editors) Molecular Industrial Mycology. Systems and Applications for Filamentous Fungi. Marcel Dekker Inc. New York 1991. pp 1-29) and Turner G. 1994 (Vectors for genetic manipulation. In: Martinelli S. D., Kinghorn J. R. (Editors) Aspergillus: 50 years on. Progress in industrial microbiology vol 29. Elsevier Amsterdam 1994. pp. 641-666). However, the following commentary provides a summary of those teachings for producing transgenic Aspergillus.

For almost a century, filamentous fungi have been widely used in many types of industry for the production of organic compounds and enzymes. For example, traditional japanese koji and soy fermentations have used Aspergillus sp. Also, in this century Aspergillus niger has been used for production of organic acids particular citric acid and for production of various enzymes for use in industry.

There are two major reasons why filamentous fungi have been so widely used in industry. First filamentous fungi can produce high amounts of extracelluar products, for example enzymes and organic compounds such as antibiotics or organic acids. Second filamentous fungi can grow on low cost substrates such as grains, bran, beet pulp etc. The same reasons have made filamentous fungi attractive organisms as hosts for heterologous expression for recombinant PME.

In order to prepare the transgenic Aspergillus, expression constructs are prepared by inserting a requisite nucleotide sequence into a construct designed for expression in filamentous fungi.

Several types of constructs used for heterologous expression have been developed. These constructs can contain a promoter which is active in fungi. Examples of promoters include a fungal promoter for a highly expressed extracelluar enzyme, such as the glucoamylase promoter or the α-amylase promoter. The nucleotide sequence can be fused to a signal sequence which directs the protein encoded by the nucleotide sequence to be secreted. Usually a signal sequence of fungal origin is used. A terminator active in fungi ends the expression system.

Another type of expression system has been developed in fungi where the nucleotide sequence can be fused to a smaller or a larger part of a fungal gene encoding a stable protein. This can stabilize the protein encoded by the nucleotide sequence. In such a system a cleavage site, recognized by a specific protease, can be introduced between the fungal protein and the protein encoded by the nucleotide sequence, so the produced fusion protein can be cleaved at this position by the specific protease thus liberating the protein encoded by the nucleotide sequence. By way of example, one can introduce a site which is recognized by a KEX-2 like peptidase found in at least some Aspergilli. Such a fusion leads to cleavage in vivo resulting in protection of the expressed product and not a larger fusion protein.

Heterologous expression in Aspergillus has been reported for several genes coding for bacterial, fungal, vertebrate and plant proteins. The proteins can be deposited intracellularly if the nucleotide sequence is not fused to a signal sequence. Such proteins will accumulate in the cytoplasm and will usually not be glycosylated which can be an advantage for some bacterial proteins. If the nucleotide sequence is equipped with a signal sequence the protein will accumulate extracelluarly.

With regard to product stability and host strain modifications, some heterologous proteins are not very stable when they are secreted into the culture fluid of fungi. Most fungi produce several extracelluar proteases which degrade heterologous proteins. To avoid this problem special fungal strains with reduced protease production have been used as host for heterologous production.

For the transformation of filamentous fungi, several transformation protocols have been developed for many filamentous fungi (Ballance 1991, ibid). Many of them are based on preparation of protoplasts and introduction of DNA into the protoplasts using PEG and Ca²⁺ ions. The transformed protoplasts then regenerate and the transformed fungi are selected using various selective markers. Among the markers used for transformation are a number of auxotrophic markers such as argB, trpC, niad and pyrG, antibiotic resistance markers such as benomyl resistance, hygromycin resistance and phleomycin resistance. A commonly used transformation marker is the amdS gene of A. nidulans which in high copy number allows the fungus to grow with acrylamide as the sole nitrogen source.

In another embodiment the transgenic organism can be a yeast. In this regard, yeast have also been widely used as a vehicle for heterologous gene expression. The species Saccharomyces cerevisiae has a long history of industrial use, including its use for heterologous gene expression. Expression of heterologous genes in Saccharomyces cerevisiae has been reviewed by Goodey et al (1987, Yeast Biotechnology, D R Berry et al, eds, pp 401429, Allen and Unwin, London) and by King et al (1989, Molecular and Cell Biology of Yeasts, E F Walton and G T Yarronton, eds, pp 107-133, Blackie, Glasgow).

For several reasons Saccharomyces cerevisiae is well suited for heterologous gene expression. First, it is non-pathogenic to humans and it is incapable of producing certain endotoxins. Second, it has a long history of safe use following centuries of commercial exploitation for various purposes. This has led to wide public acceptability. Third, the extensive commercial use and research devoted to the organism has resulted in a wealth of knowledge about the genetics and physiology as well as large-scale fermentation characteristics of Saccharomyces cerevisiae.

A review of the principles of heterologous gene expression in Saccharomyces cerevisiae and secretion of gene products is given by E Hinchcliffe E Kenny (1993, “Yeast as a vehicle for the expression of heterologous genes”, Yeasts, Vol 5, Anthony H Rose and J Stuart Harrison, eds, 2nd edition, Academic Press Ltd.).

Several types of yeast vectors are available, including integrative vectors, which require recombination with the host genome for their maintenance, and autonomously replicating plasmid vectors.

In order to prepare the transgenic Saccharomyces, expression constructs are prepared by inserting the nucleotide sequence into a construct designed for expression in yeast. Several types of constructs used for heterologous expression have been developed. The constructs contain a promoter active in yeast fused to the nucleotide sequence, usually a promoter of yeast origin, such as the GALL promoter, is used. Usually a signal sequence of yeast origin, such as the sequence encoding the SUC2 signal peptide, is used. A terminator active in yeast ends the expression system.

For the transformation of yeast several transformation protocols have been developed. For example, a transgenic Saccharomyces can be prepared by following the teachings of Hinnen et al (1978, Proceedings of the National Academy of Sciences of the USA 75, 1929); Beggs, J D (1978, Nature, London, 275, 104); and Ito, H et al (1983, J Bacteriology 153, 163-168).

The transformed yeast cells are selected using various selective markers. Among the markers used for transformation are a number of auxotrophic markers such as LEU2, HIS4 and TRP1, and dominant antibiotic resistance markers such as aminoglycoside antibiotic markers, eg G418.

Another host organism is a plant. In this regard, the art is replete with references for preparing transgenic plants. Two documents that provide some background commentary on the types of techniques that may be employed to prepare transgenic plants are EP-B-0470145 and CA-A-2006454—some of which commentary is presented below.

The basic principle in the construction of genetically modified plants is to insert genetic information in the plant genome so as to obtain a stable maintenance of the inserted genetic material.

Several techniques exist for inserting the genetic information, the two main principles being direct introduction of the genetic information and indirect introduction of the genetic information by use of a vector system. A review of the general techniques may be found in articles by Potrykus (Annu Rev Plant Physiol Plant Mol Biol [1991] 42:205-225) and Christou (Agro-Food-Industry Hi-Tech March/April 1994 17-27).

A suitable transformation system for a plant may comprise one vector, but it can comprise two vectors. In the case of two vectors, the vector system is normally referred to as a binary vector system. Binary vector systems are described in further detail in Gynheung An et al. (1980), Binary Vectors, Plant Molecular Biology Manual A3, 1-19.

One extensively employed system for transformation of plant cells with a given promoter or nucleotide sequence or construct is based on the use of a Ti plasmid from Agrobacterium tumefaciens or a Ri plasmid from Agrobacterium rhizogenes as described in An et al. (1986), Plant Physiol. 81, 301-305 and Butcher D. N. et al. (1980), Tissue Culture Methods for Plant Pathologists, eds.: D. S. Ingrams and J. P. Helgeson, 203-208.

Several different Ti and Ri plasmids have been constructed which are suitable for the construction of the plant or plant cell constructs described above. A non-limiting example of such a Ti plasmid is pGV3850.

The nucleotide sequence or construct should preferably be inserted into the Ti-plasmid between the terminal sequences of the T-DNA or adjacent a T-DNA sequence so as to avoid disruption of the sequences immediately surrounding the T-DNA borders, as at least one of these regions appear to be essential for insertion of modified T-DNA into the plant genome.

As will be understood from the above explanation, if the organism is a plant, then the vector system is preferably one which contains the sequences necessary to infect the plant (e.g. the vir region) and at least one border part of a T-DNA sequence, the border part being located on the same vector as the genetic construct. Preferably, the vector system is an Agrobacterium tumefaciens Ti-plasmid or an Agrobacterium rhizogenes R1-plasmid or a derivative thereof, as these plasmids are well-known and widely employed in the construction of transgenic plants, many vector systems exist which are based on these plasmids or derivatives thereof.

In the construction of a transgenic plant the nucleotide sequence may be first constructed in a microorganism in which the vector can replicate and which is easy to manipulate before insertion into the plant. An example of a useful microorganism is E. coli., but other microorganisms having the above properties may be used. When a vector of a vector system as defined above has been constructed in E. coli. it is transferred, if necessary, into a suitable Agrobacterium strain, e.g. Agrobacterium tumefaciens. The Ti-plasmid harbouring the nucleotide sequence or construct is thus preferably transferred into a suitable Agrobacterium strain, e.g. A. tumefaciens, so as to obtain an Agrobacterium cell harbouring the nucleotide sequence, which DNA is subsequently transferred into the plant cell to be modified.

As reported in CA-A-2006454, a large amount of cloning vectors are available which contain a replication system in E. coli and a marker which allows a selection of the transformed cells. The vectors contain for example pBR 322, the pUC series, the M13 mp series, pACYC 184 etc.

In this way, the nucleotide sequence can be introduced into a suitable restriction position in the vector. The contained plasmid is used for the transformation in E. coli. The E. coli cells are cultivated in a suitable nutrient medium and then harvested and lysed. The plasmid is then recovered. As a method of analysis there is generally used sequence analysis, restriction analysis, electrophoresis and further biochemical-molecular biological methods. After each manipulation, the used DNA sequence can be restricted and connected with the next DNA sequence. Each sequence can be cloned in the same or different plasmid.

After each introduction method of the desired promoter or construct or nucleotide sequence in the plants the presence and/or insertion of further DNA sequences may be necessary. If, for example, for the transformation the Ti- or Ri-plasmid of the plant cells is used, at least the right boundary and often however the right and the left boundary of the Ti- and Ri-plasmid T-DNA, as flanking areas of the introduced genes, can be connected. The use of T-DNA for the transformation of plant cells has been intensively studied and is described in EP-A-120516; Hoekema, in: The Binary Plant Vector System Offset-drukkerij Kanters B. B., Alblasserdam, 1985, Chapter V; Fraley, et al., Crit. Rev. Plant Sci., 4:146; and An et al., EMBO J. (1985) 4:277-284.

Direct infection of plant tissues by Agrobacterium is a simple technique which has been widely employed and which is described in Butcher D. N. et al. (1980), Tissue Culture Methods for Plant Pathologists, eds.: D. S. Ingrams and J. P. Helgeson, 203-208. For further teachings on this topic see Potrykus (Annu Rev Plant Physiol Plant Mol Biol [1991] 42:205-225) and Christou (Agro-Food-Industry Hi-Tech March/April 1994 17-27). With this technique, infection of a plant may be done on a certain part or tissue of the plant, i.e. on a part of a leaf, a root, a stem or another part of the plant.

Typically, with direct infection of plant tissues by Agrobacterium carrying the promoter and the nucleotide sequence of the present invention, a plant to be infected is wounded, e.g. by cutting the plant with a razor or puncturing the plant with a needle or rubbing the plant with an abrasive. The wound is then inoculated with the Agrobacterium. The inoculated plant or plant part is then grown on a suitable culture medium and allowed to develop into mature plants.

When plant cells are constructed, these cells may be grown and maintained in accordance with well-known tissue culturing methods such as by culturing the cells in a suitable culture medium supplied with the necessary growth factors such as amino acids, plant hormones, vitamins, etc. Regeneration of the transformed cells into genetically modified plants may be accomplished using known methods for the regeneration of plants from cell or tissue cultures, for example by selecting transformed shoots using an antibiotic and by subculturing the shoots on a medium containing the appropriate nutrients, plant hormones, etc.

Another technique for transforming plants is ballistic transformation. Originally developed to produce stable transformants of plant species which were recalcitrant to transformation by Agrobacterium tumefaciens, ballistic transformation of plant tissue, which introduces DNA into cells on the surface of metal particles, has found utility in testing the performance of genetic constructs during transient expression. In this way, gene expression can be studied in transiently transformed cells, without stable integration of the gene in interest, and thereby without time-consuming generation of stable transformants.

In more detail, the ballistic transformation technique (otherwise known as the particle bombardment technique) was first described by Klein et al. [1987], Sanford et al. [1987] and Klein et al. [1988] and has become widespread due to easy handling and the lack of pre-treatment of the cells or tissue in interest.

The principle of the particle bombardment technique is direct delivery of DNA-coated microprojectiles into intact plant cells by a driving force (e.g. electrical discharge or compressed air). The microprojectiles penetrate the cell wall and membrane, with only minor damage, and the transformed cells then express the promoter constructs.

One particle bombardment technique that can be performed uses the Particle Inflow Gun (PIG), which was developed and described by Finer et al. [1992] and Vain et al. [1993]. The PIG accelerates the microprojectiles in a stream of flowing helium, through a partial vacuum, into the plant cells.

One of advantages of the PIG is that the acceleration of the microprojectiles can be controlled by a timer-relay solenoid and by regulation the provided helium pressure. The use of pressurised helium as a driving force has the advantage of being inert, leaves no residues and gives reproducible acceleration. The vacuum reduces the drag on the particles and lessens tissue damage by dispersion of the helium gas prior to impact [Finer et al. 1992].

Other techniques for transforming plants include the silicon whisker technique and viral transformation techniques.

Further teachings on plant transformation may be found in EP-A-0449375, U.S. Pat. No. 5,387,757, U.S. Pat. No. 5,569,831, U.S. Pat. No. 5,107,065, EP-A-0341885, EP-A-0271988, EP-A-0416572, EP-A-0240208, EP-A-0458367, WO-A-97/37023, WO-A-94/21803, WO-A-93/23551, WO-A-95/23227.

Even though the amino acid sequence of formula (I) is believed to play an important role in the block-wise de-esterifaction properties of a PME, we also believe that the sequence may also affect the enzymatic activity of other enzymes if it is present in the sequence for those other enzymes. For example, the amino acid sequence of formula (I) may be introduced into enzymes such as pectin acetylesterase or rhamnogalacturonan acetylesterase. In this respect, the presence of the amino acid sequence of formula (I) might yield an acetylesterase which is capable of de-acetylating blockwise (e.g. the sugar beet pectin or the “hairy region” in the several pectins, respectively). The amino acid sequence of formula (I) may even be introduced into enzymes such as xylan acetylesterase. In this respect, the presence of the amino acid sequence of formula (I) might yield an acetylesterase which is capable of de-acetylating xylan in a blockwise manner. Hence, each of the above-mentioned embodiments of the present invention relating to a modified PME may also be applicable to a modified enzyme in the general sense.

As indicated above, the present invention also encompasses homologues of the presented sequences. As also indicated, the degree of homology (or identity) can be determined by a simple “eyeball” comparison (i.e. a strict comparison) of any one or more of the sequences with another sequence or by use commercially available computer programs that can calculate % homology between two or more sequences.

If a commercial program is used, the sequence homology (or identity) can be determined using any suitable homology algorithm, using for example default parameters. Advantageously, the BLAST algorithm is employed, with parameters set to default values. The BLAST algorithm is described in detail at http://www.ncbi.nih.gov/BLAST/blast_help.html, which is incorporated herein by reference. The search parameters are defined as follows, and are advantageously set to the defined default parameters.

Advantageously, “substantial homology” when assessed by BLAST equates to sequences which match with an EXPECT value of at least about 7, preferably at least about 9 and most preferably 10 or more. The default threshold for EXPECT in BLAST searching is usually 10.

BLAST (Basic Local Alignment Search Tool) is the heuristic search algorithm employed by the programs blastp, blastn, blastx, tblastn, and tblastx; these programs ascribe significance to their findings using the statistical methods of Karlin and Altschul (see htt p://www.ncbi.nih.gov/BLAST/blast_help.html) with a few enhancements. The BLAST programs were tailored for sequence similarity searching, for example to identify homologues to a query sequence. The programs are not generally useful for motif-style searching. For a discussion of basic issues in similarity searching of sequence databases, see Altschul et al (1994) Nature Genetics 6:119-129.

The five BLAST programs available at http://www.ncbi.nlm.nih.gov perform the following tasks:

blastp compares an amino acid query sequence against a protein sequence database;

blastn compares a nucleotide query sequence against a nucleotide sequence database;

blastx compares the six-frame conceptual translation products of a nucleotide query sequence (both strands) against a protein sequence database;

tblastn compares a protein query sequence against a nucleotide sequence database dynamically translated in all six reading frames (both strands).

tblastx compares the six-frame translations of a nucleotide query sequence against the six-frame translations of a nucleotide sequence database.

BLAST uses the following search parameters:

HISTOGRAM Display a histogram of scores for each search; default is yes. (See parameter H in the BLAST Manual).

DESCRIPTIONS Restricts the number of short descriptions of matching sequences reported to the number specified; default limit is 100 descriptions. (See parameter V in the manual page). See also EXPECT and CUTOFF.

ALIGNMENTS Restricts database sequences to the number specified for which high-scoring segment pairs (HSPs) are reported; the default limit is 50. If more database sequences than this happen to satisfy the statistical significance threshold for reporting (see EXPECT and CUTOFF below), only the matches ascribed the greatest statistical significance are reported. (See parameter B in the BLAST Manual).

EXPECT The statistical significance threshold for reporting matches against database sequences; the default value is 10, such that 10 matches are expected to be found merely by chance, according to the stochastic model of Karlin and Altschul (1990). If the statistical significance ascribed to a match is greater than the EXPECT threshold, the match will not be reported. Lower EXPECT thresholds are more stringent, leading to fewer chance matches being reported. Fractional values are acceptable. (See parameter E in the BLAST Manual).

CUTOFF Cutoff score for reporting high-scoring segment pairs. The default value is calculated from the EXPECT value (see above). HSPs are reported for a database sequence only if the statistical significance ascribed to them is at least as high as would be ascribed to a lone HSP having a score equal to the CUTOFF value. Higher CUTOFF values are more stringent, leading to fewer chance matches being reported. (See parameter S in the BLAST Manual). Typically, significance thresholds can be more intuitively managed using EXPECT.

MATRIX Specify an alternate scoring matrix for BLASTP, BLASTX, TBLASTN and TBLASTX. The default matrix is BLOSUM62 (Henikoff & Henikoff, 1992). The valid alternative choices include: PAM40, PAM120, PAM250 and IDENTITY. No alternate scoring matrices are available for BLASTN; specifying the MATRIX directive in BLASTN requests returns an error response.

STRAND Restrict a TBLASTN search to just the top or bottom strand of the database sequences; or restrict a BLASTN, BLASTX or TBLASTX search to just reading frames on the top or bottom strand of the query sequence.

FILTER Mask off segments of the query sequence that have low compositional complexity, as determined by the SEG program of Wootton & Federhen (1993) Computers and Chemistry 17:149-163, or segments consisting of short-periodicity internal repeats, as determined by the XNU program of Clayerie & States (1993) Computers and Chemistry 17:191-201, or, for BLASTN, by the DUST program of Tatusov and Lipman (see http://www.ncbi.nlm.nih.gov). Filtering can eliminate statistically significant but biologically uninteresting reports from the blast output (e.g., hits against common acidic-, basic- or proline-rich regions), leaving the more biologically interesting regions of the query sequence available for specific matching against database sequences.

Low complexity sequence found by a filter program is substituted using the letter “N” in nucleotide sequence (e.g., “NNNNNNNNNNNNN”) and the letter “X” in protein sequences (e.g., “XXXXXXXXX”).

Filtering is only applied to the query sequence (or its translation products), not to database sequences. Default filtering is DUST for BLASTN, SEG for other programs.

It is not unusual for nothing at all to be masked by SEG, XNU, or both, when applied to sequences in SWISS-PROT, so filtering should not be expected to always yield an effect. Furthermore, in some cases, sequences are masked in their entirety, indicating that the statistical significance of any matches reported against the unfiltered query sequence should be suspect.

NCBI-gi Causes NCBI gi identifiers to be shown in the output, in addition to the accession and/or locus name.

Most preferably, sequence comparisons are conducted using the simple BLAST search algorithm provided at http://www. ncbi. nlm. nih. gov/BLAST.

Other computer program methods to determine identify and similarity between the two sequences include but are not limited to the GCG program package (Devereux et al 1984 Nucleic Acids Research 12: 387 and FASTA (Atschul et al 1990 J Molec Biol 403-410).

Should Gap Penalties be used when determining sequence identity, then preferably the following parameters are used:

| FOR BLAST | |
|---|---|
| GAP OPEN | 0 |
| GAP EXTENSION | 0 |
| FOR CLUSTAL | DNA |
| WORD SIZE | 2 |
| GAP PENALTY | 10 |
| GAP EXTENSION | 0.1 |

As used herein, the terms “variant”, “homologue”, “fragment” and “deriavtive” embrace allelic variations of the sequences.

The term “variant” also encompasses sequences that are complementary to sequences that are capable of hydridising to the nucleotide sequences presented herein.

In some instances, it is desirable to position a trytophan between A2 and A3 in formula (1). In this embodiment, the tryptophan would actually become position No. 3. However, the ordering of the consequential amino acids remains the same. For convenience we shall call this modification of formula (I), formula (IA).

Thus, in one aspect, the present invention also encompasses an amino acid sequence of the formula (IA):

A1-A2-W-A3-A4-A5-A6-A7-A8-A9-A10-A11-A12-A13-A14-A15-A16-A17-A18-A19-A20-A21-A22 (IA)

wherein

W represents tryptophan

A1 is a hydrophobic or polar amino acid or a neutral amino acid

A2 is a hydrophobic amino acid

A3 is a hydrophobic amino acid

A4 is a polar amino acid

A5 is a polar or charged amino acid or a neutral amino acid

A6 is a polar amino acid

A7 is a polar or charged or hydrophobic amino acid

A8 is a hydrophobic amino acid

A9 is a hydrophobic or polar amino acid

A10 is a hydrophobic or polar amino acid

A11 is a charged amino acid

A12 is a charged or polar or hydrophobic amino acid

A13 is a hydrophobic or charged amino acid or a neutral amino acid

A14 is a hydrophobic or polar amino acid or charged or neutral amino acid

A15 is a charged or polar or hydrophobic amino acid

A16 is a polar or hydrophobic or charged amino acid or a neutral amino acid

A17 is a polar or charged amino acid a neutral amino acid

A18 is a polar or charged or hydrophobic amino acid

A19 is a polar amino acid or a neutral amino acid

A20 is a hydrophobic or polar amino acid

A21 is a hydrophobic amino acid

A22 is a polar or hydrophobic amino acid.

In this respect, all of the teachings relating to formula (I) and its preferred aspects are equally applicable to formula (IA).

By way of example, N terminal sequences of examples covered by formula (IA) include:

RAWFHECDI . . .

AVWFQNCDI . . .

AVWFQNCDI . . .

In addition, or in the alternative, A9 and/or A10 and/or A22 can be omitted. For convenience we shall call this modification of formula (I) and/or formula (IA), formula (IB).

Thus, in one aspect, the present invention also encompasses an amino acid sequence of the formula (IB):

A1-A2-W-A3-A4-A5-A6-A7-A8-A9-A10-A11-A12-A 13-A 14-A15-A16-A17-A18-A 19-A20-A21-A22 (IB)

wherein

W represents an optional tryptophan

A1 is a hydrophobic or polar amino acid or a neutral amino acid

A2 is a hydrophobic amino acid

A3 is a hydrophobic amino acid

A4 is a polar amino acid

A5 is a polar or charged amino acid or a neutral amino acid

A6 is a polar amino acid

A7 is a polar or charged or hydrophobic amino acid

A8 is a hydrophobic amino acid

A9 is an optional hydrophobic or an optional polar amino acid

A10 is an optional hydrophobic or an optional polar amino acid

A11 is a charged amino acid

A12 is a charged or polar or hydrophobic amino acid

A13 is a hydrophobic or charged amino acid or a neutral amino acid

A14 is a hydrophobic or polar amino acid or charged or neutral amino acid

A15 is a charged or polar or hydrophobic amino acid

A16 is a polar or hydrophobic or charged amino acid or a neutral amino acid

A17 is a polar or charged amino acid a neutral amino acid

A18 is a polar or charged or hydrophobic amino acid

A19 is a polar amino acid or a neutral amino acid

A20 is a hydrophobic or polar amino acid

A21 is a hydrophobic amino acid

A22 is an optional polar amino acid or an optional hydrophobic amino acid.

In this respect, all of the teachings relating to formula (I) and its preferred aspects are equally applicable to formula (IB).

By way of example, examples of sequences covered by formula (IB) include:

AV-FQNCDTHARKPNDGQKNMV
AVWFQNCDIHARKPNDGQKNMV
AVWFQNCDI--RKPNDGQKNMV
AV-FQNCDIHARKPNDGQKNMV

The present invention will now be described only by way of examples.

### EXAMPLE 1

The nucleotide sequence coding for the amino acid sequence of formula (I)—such as that presented below—is introduced into a gene coding for a PME that does not exhibit block-wise de-esterification properties—such as the PME from Aspergillus niger.

Sequence for insertion:

GCCGTGTTACAAAATTGTGACATCCATGCACGAAAGCCCAATTCCGGCCA
AAAAAATATGGTCACA

This sequence can be a synthetic sequence or it can be produced by use of recombinant DNA techniques.

The positioning of the sequence is near to the 3′ end of the gene portion coding the PME active site.

A 66 nucleotide sequence is removed next to the insertion site.

The resultant modified PME from Aspergillus niger is then produced by, for example, transforming Aspergillus by suitably adapting the above teachings and references for Aspergillus transformation. The modified PME is then used to modify a pectin by bringing the pectin into contact with the modified PME in a suitable reaction environment. The modified PME sample can be an isolated and/or pure sample or it can be a crude extract.

The block-wise de-esterification properties PME and the properties of a pectin treated by same may be determined by the Protocols mentioned below.

Surprisingly, the expressed modified PME exhibits a different PME profile, in particular it exhibits at least some block-wise de-esterification properties (i.e. at least a partial block-wise de-esterification property).

### EXAMPLE 2

The nucleotide sequence coding for the amino acid sequence of formula (I) is removed from a gene that codes for a PME that exhibits block-wise de-esterification properties—such as the PME from orange.

The sequence to be removed is

GCCGTGTTACAAAATTGTGACATCCATGCACGAAAGCCCAATTCCGGCCA
AAAAAATATGGTCACA.

A 66 nucleotide sequence is then inserted into the removal site. This 66 nucleotide sequence does not code for an amino acid sequence of formula (I).

The resultant modified PME from orange is then produced by, for example, transforming a suitable host cell—such as a plant cell—by suitably adapting the above teachings and references for plant transformation. The modified PME is then used to modify a pectin by bringing the pectin into contact with the modified PME in a suitable reaction environment. The modified PME sample can be an isolated and/or pure sample or it can be a crude extract.

The random de-esterification properties of the PME and the properties of a pectin treated by same may be determined by the Protocols mentioned below.

Surprisingly, the expressed modified PME exhibits a different PME profile, in particular it exhibits random de-esterification properties.

### EXAMPLE 3

The nucleotide sequence coding for the amino acid sequence of formula (I) is removed from a gene that codes for a PME that exhibits block-wise de-esterification properties—such as the PME from a tomato.

The sequence to be removed is

GCCGTGTTACAAAATTGTGACATCCATGCACGAAAGCCCAATTCCGGCCA
AAAAAATATGGTCACA.

A 66 nucleotide sequence is then inserted into the removal site. This 66 nucleotide sequence does not code for an amino acid sequence of formula (I).

The resultant modified PME from tomato is then produced by, for example, transforming a suitable host cell—such as a plant cell—by suitably adapting the above teachings and references for plant transformation. The modified PME is then used to modify a pectin by bringing the pectin into contact with the modified PME in a suitable reaction environment. The modified PME sample can be an isolated and/or pure sample or it can be a crude extract.

The random de-esterification properties of the PME and the properties of a pectin treated by same may be determined by the Protocols mentioned below.

Surprisingly, the expressed modified PME exhibits a different PME profile, in particular it exhibits random de-esterification properties.

In this example, expression of the modified PME will be achieved by utilising the Cauliflower Mosaic Virus (CaMV) ³⁵S promoter into various plant types, such as tomato genotypes. This highly expressed constitutive promoter is widely available. Other promoters may also be utilized. The constitutive CaMV ³⁵S promoter will be initially used for the proposed experiments because this promoter has been shown to promote high levels of protein production in most plant organs, including tomato fruit.

First, an DNA construction is created—which comprises the nucleotide sequence coding for the modified PME. At a minimum, this DNA construction contains a promoter effective to promote transcription in tomato plants, a cDNA clone encoding the modified PME, and a sequence effective to terminate transcription. Using standard molecular biological methods, the CaMV35S promoter sequence will be attached to the encoding sequence. A suitable termination sequence, such as the nopaline synthase 3′ terminator, will be placed downstream from the cDNA insert. The DNA construction will be placed in an appropriate vector for plant transformation. For Agrobacterium-mediated transformation, the promoter/cDNA/terminator construction will preferably be placed in a Ti-based plasmid, such as pBI121, a standard binary vector. In general, transformation will preferably be done with two standard Agrobacterium binary vectors: pBI121 (sold by Clontech Laboratories, Palo Alto Calif.) and pGA643 (developed by G. An at Washington State University). pBI121 contains a CAMV promoter and GUS reporter gene. The GUS coding sequence will be removed by digesting with SstI and SmaI (blunt end). The modified PME coding sequence to be used could be produced by digesting with with appropriate restriction enzymes. The sticky/blunt ends will allow for directional cloning into pBI121. Standard methods for cutting, ligating and E. coli transformation will be used.

For plant transformation, it is possible to follow, in general, the methods of McCormick (1986, Plant Cell Reporter 5:81-84) and Plant Tissue Culture Manual B6:1-9 (1991) Kluwer Academic Publishers. This later reference compiles/compares various procedures for Agrobacterium-mediated transformation of tomato.

### Protocols

### Protocol I

### Calcium Sensitivity Index (CF)

Calcium sensitivity is measured as the viscosity of a pectin dissolved in a solution with 57.6 mg calcium/g pectin divided by the viscosity of exactly the same amount of pectin in solution, but without added calcium. A calcium insensitive pectin has a CF value of 1.

4.2 g pectin sample is dissolved in 550 ml hot water with efficient stirring. The solution is cooled to about 20° C. and the pH adjusted to 1.5 with 1N HCl. The pectin solution is adjusted to 700 ml with water and stirred. 145 g of this solution is measured individually into 4 viscosity glasses. 10 ml water is added to two of the glasses (double determinations) and 10 ml of a 250 mM CaCl₂ solution is added to the other two glasses under stirring.

50 ml of an acetate buffer (0.5 M, pH about 4.6) is added to all four viscosity glasses under efficient magnetic stirring, thereby bringing the pH of the pectin solution up over pH 4.0. The magnets are removed and the glasses left overnight at 20° C. The viscosities are measured the next day with a Brookfield viscometer. The calcium sensitivity index is calculated as follows:

$$CF = \frac{Viscosity of a solution with 57.6 mg {Ca}^{2 +} / g pectin}{Viscosity of a solution with 0.0 mg {Ca}^{2 +} / g pectin}$$

### Protocol II

### Degree of Esterification (%DE)

To 50 ml of a 60% isopropanol and a 5% HCl solution is added 2.5 g pectin sample and stirred for 10 min. The pectin solution is filtered through a glass filter and washed with 15 ml 60% isopropanol/S % HCl solution 6 times followed by further washes with 60% isopropanol until the filtrate is free of chlorides. The filtrate is dried overnight at 80° C.

20.0 ml 0.5 N NaOH and 20.0 ml 0.5 N HCl is combined in a conical flask and 2 drops of phenolphtalein is added. This is titrated with 0.1 N NaOH until a permanent colour change is obtained. The 0.5 N HCl should be slightly stronger than the 0.5N NaOH. The added volume of 0.1 N NaOH is noted as V₀.

0.5 g of the dried pectin sample (the filtrate) is measured into a conical flask and the sample is moistened with 96% ethanol. 100 ml of recently boiled and cooled destined water is added and the resulting solution stirred until the pectin is completely dissolved. Then 5 drops of phenolphtalein are added and the solution titrated with 0.1 N NaOH (until a change in colour and pH is 8.5). The amount of 0.1 N NaOH used here is noted as V₁. 20.0 ml of 0.5 N NaOH is added and the flask shaken vigously, and then allowed to stand for 15 min. 20.0 ml of 0.5 N HCl is added and the flask is shaken until the pink colour disappears. 3 drops of phenolphtalein are then added and then the resultant solution is titrated with 0.1 N NaOH. The volume 0.1 N NaOH used is noted as V₂.

The degree of esterification (% DE: % of total carboxy groups) is calculated as follows:

$$\% DE = \frac{{V}_{2} - {V}_{0}}{{V}_{1} + ( {V}_{2} - {V}_{0} )}$$

### Protocol III

### Drink Test

A Small Scale Method for Screening Pectins in an Acidified Milk Drink System

1. Introduction

Acidified milk drinks with long shelf life are very popular, especially in the Far East. A heat treatment is necessary to obtain a long shelf life, and in order to avoid sedimentation of protein during and after heating, pectin is added as a stabilising agent. As the quality of the acidified milk drink depends strongly on the properties and the concentration of the pectin used, the effect of pectin stabilisation has been investigated in different model systems.

KRAVTCHENKO et al. (1) used commercial yoghurt as a base. The yoghurt was homogenised, and a pectin solution was added, without any following heat treatment. GLAHN (2) acidified reconstituted skim milk powder with glucono-d-lactone (GDL). After addition of pectin dispersed in sugar, the mixture was homogenised, heat-treated and homogenised a second time. Almost the same procedure was used by FOLEY AND MULCAHY (3), although they omitted the last homogenisation. AMICE-QUEMENEUR et al. (4) also used reconstituted skim milk powder acidified with either GDL or yoghurt culture. The yoghurt base was added a solution of pectin in water, and homogenised with an Ultra-Turrax, while no heat treatment was applied. PEDERSEN AND JØRGENSEN (5) used an aqueous mixture of pectin and casein without any homogenisation or heat treatment.

Most of the systems used in these studies require fairly large amounts of pectin. Another limitation is that in most cases only one type of pectin was used. Since the stabilisation power of pectin is very dependent on the chemical structure and functional properties the same test made with other types of pectin might lead to different conclusions, regarding the mechanisms involved in stabilisation of milk proteins. It is therefore valuable to establish a system that allows many samples of pectins (e.g. experimental laboratory samples) to be tested. Since laboratory production of pectins normally yield very small amounts of sample, is it important that such a model system only requires a small amount of pectin.

The following describes a protocol that only uses about 1.7 g pectin to as little as possible. The methods used to evaluate the performance of the system were viscometry, centrifugal sedimentation, and particle size determination.

2. Materials and Methods

2.1 Materials

Skim milk powder with approx. 36% protein was obtained from Mejeriernes Faexlles Indkøb (Kolding, Denmark). Pectins for testing were obtained by treatment of a pectin with a modified PME according to the present invention. These pectins may have different properties such as degree of esterification and molecular weight, depending on the type of modified PME used.

2.2 Preparation of Milk Drink

The milk drinks were made by mixing an acidified milk solution and a pectin solution, followed by further processing.

A milk solution was made by dissolving 17% (w/w) skimmilk powder in distilled water at 68° C. and stirring for 30 min. The milk solution was then acidified to pH 4.1 at 30° C. by addition of 3% (w/w) glucono-d-lactone (GDL).

The pectin solution was made up in several steps. First pectin was dry mixed with dextrose at a 3:2 weight ratio, and then a 1.11% (w/w) solution of this mixture in distilled water was made. The last step in the preparation of the pectin solution was to add sucrose to an end concentration of 17.8% (w/w).

Milk drinks were then prepared by mixing 1 part of milk solution with 1.13 parts (w/w) of pectin solution, followed by heat treatment (see section 3.2) and homogenisation at 20-22 MPa and 20° C. using a Mini Jet Homogeniser (Burgaud et. al., 1990). By following this procedure,the final concentration of pectin in the milk drink was 0.3% (w/w). All samples were produced in duplicate, stored at 5° C. and tested for viscosity, particle size and sedimentation the following day.

2.3 Viscosity Measurement

The viscosity was measured using a Bohlin VOR Rheometer system (Bohlin Instruments, Metric Group Ltd., Gloucestershire, Great Britain). Thermostatation was achieved by a Bohlin lower-plate temperature control unit. The viscosity was measured at a shear rate of 91.9 s⁻¹. The measuring temperature was 20° C., and the samples were held at 20° C. for approximately 1 hour before measurement. The measuring system used was C 14 (a coaxial cylindrical system). The torque element used was 0.25 g cm. Integration time was 5 s, measurement interval was 30 s, and no autozero was used. Instrumental control and primary data processing were done on a PC with the Bohlin Rheometer Software version 4.05.

2.4 Particle Size Measurement

The particle mean diameter, D[4.3], was measured with a Malvern Mastersizer Micro Plus (Malvern Instruments Limited, Worcestershire, UK). Instrumental settings were: presentation code: 5NBD, and Analysis Model: polydisperse. Instrumental control and primary data processing were done on a PC with Mastersizer Microplus for Windows, version 2.15.

Ultrafiltration permeate obtained from a batch of acidified milk drink made with pectin no. 4 was used for dilution. Ultrafiltration was done using a DDS UF Lab 20-0.36 module fitted with GR61PP membranes, having a molecular weight cut-off of 20.000 Da.

2.5 Sedimentation

Sedimentation measurements were performed by centrifugation of the samples using an IEC Centra-8R Centrifuge (International Equipment, Needham Hts, Mass., USA). 2.5 g acidified milk drink was centrifuged for 25 min at 20° C. and 2400 g. The supernatant was removed, the tubes were left up side down for 15 min, and the weight of the sediment was determined and expressed as a percentage (of the amount of milk drink used). Duplicate measurements were made of each sample.

3. Results and Discussion

3.1 Size of Test System

This new system is small compared to the previous test systems but it still maintains the same properties as the existing test system based on 550 g acidified milk drink. The easiest way to make a model system for testing pectins in acidified milk drinks would be to simply mix stirred yoghurt with α-pectin solution, and make the measurements on this mixture. This also has the advantage that it can be done virtually at any scale. However, GLAHN AND ROLIN (6) showed that a homogenisation reduces the amount of pectin needed for stabilisation and that both homogenisation and heat treatment have very considerable effects on stability. Since both homogenisation and heat treatment were included in the existing system at 550 g scale, as they are in industrial processes, both treatments also needed to be present in the small scale system. In industry both upstream (before heating) and downstream (after heating) homogenisation is used. In this model system we chose to put the homogenisation in after heat treatment because this gives a more homogeneous sample, and thereby makes it easier to obtain reproducible measurements of e.g. viscosity.

To achieve a reproducible homogenisation with the Mini Jet Homogeniser, and to compensate for various losses during sample transfer, it was desirable to operate with 40 ml of sample at the homogenisation stage. Since only 8-9 ml was needed for the tests (2.5 ml for viscometry, 5 ml for sedimentation, and 0.5-1 ml for particle size determination), the step that required the largest amount of sample was the homogenisation, and the result was therefore that the existing test system was scaled down from 550 g to 40 g milk drink.

3.2 Heat Treatment

To make the scaled down system mimic the existing test system as closely as possible it was desirable to make modifications to the heat treatment step. With the existing 550 g system heating took place in a 600 ml Blue-cap bottle for 30 min in a 75° C. water bath, with stirring every 5 minutes.

With the new 40 g system the heat treatment was done in a 50 ml plastic centrifuge tube placed inside a 600 ml Blue-cap bottle filled with water. Here 75° C. in the water bath gave too strong a heating, probably because the thermal conductivity of water is larger than that of coagulated milk. Different temperatures between 70 and 75° C. were therefore tested, and it was found that 72° C. for 30 minutes, without stirring, gave a good approximation to the temperature profile in the large system.

3.3 Testing of Small Scale System

If a milk drink stabilised with a pectin treated with a modified PME according to the present invention showed little sedimentation and small particles, then that indicates a good pectin to use and moreover is indicative that the modified PME according to the present invention is suitable for such a use.

4. Conclusion

A system for testing the stabilising power of pectins in acidified milk drinks has successfully been scaled down from 550 g to 40 g milk drink, meaning that the required amount of pectin is reduced from ca. 1.7 g to ca. 0.15 g. This is small enough to allow screening of experimental pectin samples treated with modified pectins according to the present invention. A high correlation between results obtained for particle size, viscosity and sedimentation between the two methods has been demonstrated. The scaled down method is relatively simple, although it still contains both heating and homogenisation, which is considered important for industrial relevance.

For convenience, we now present a Table indicating the codes used for the amino acids.

| | THREE LETTER | |
|---|---|---|
| AMINO ACID | ABBREVIATION | ONE LETTER SYMBOL |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any residue | Xaa | X |

All publications mentioned in the above specification are herein incorporated by reference. Various modifications and variations of the described methods and system of the present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in biochemistry and biotechnology or related fields are intended to be within the scope of the following claims.

### REFERENCES

(1) KRAVTCHENKO, T. P., PARKER, A., TRESPOEY,A.: In Food Macromolecules and Colloids (Ed. E. Dickinson and D. Lorient). The Royal Society of Chemistry, Cambridge (1995)

(2) GLAHN, P. -E.: Progress in Food Nutrient Science 6 171-177 (1982)

(3) FOLEY, J., MULCAHY, A. J.: Irish Journal of Food Science and Technology 13 43-50 (1989)

(4) AMICE-QUEMENEUR, N., HALUK, J. -P., HARDY, J.: Journal of Dairy Science 78 (12) 2683-2690 (1995)

(5) AMBJERG PEDERSEN, H. C., JØRGENSEN, B. B.: Food Hydrocolloids 5 (4) 323-328 (1997)

(6) GLAHN, P. E., ROLIN, C.: Food Ingredients Europe, Conf . Proc. 252-256 (1994)

(7) BURGAUD, I., DICKINSON, E., Nelson, E.: International Journal of Food Science and Technology 25, 39-46 (1990)

Finer J J, Vain P, Jones M W & McMullen M D (1992) Development of the particle inflow gun for DNA delivery to plant cells Plant cell Reports 11: 323-328

Klein T M, Wolf E D, Wu R & Sanford J C (1987) High-velocity microprojectiles for delivery nucleic acids into living cells Nature 327: 70-73

Sanford J C, Klein T M, Wolf E D & Allen N (1987) Delivery of substances into cells and tissues using a particle bombardment process Particulate Science and Technology 5: 27-37

Vain P, Keen N, Murillo J, Rathus C, Nemes C & Finer J J (1993) Development of the Particle Inflow Gun Plant cell, Tissue and Organ Culture 33: 237-246

1. An amino acid sequence of the formula (I):
A1-A2-A3-A4-A5-A6-A7-A8-A9-A10-A11-A12-A13-A14-A15-A16-A17-A18-A19-A20-A2 1-A22 (i)
wherein
A1 is a hydrophobic or polar amino acid or a neutral amino acid
A2 is a hydrophobic amino acid
A3 is a hydrophobic amino acid
A4 is a polar amino acid
A5 is a polar or charged amino acid or a neutral amino acid
A6 is a polar amino acid
A7 is a polar or charged or hydrophobic amino acid
A8 is a hydrophobic amino acid
A9 is a hydrophobic or polar amino acid
A10 is a hydrophobic or polar amino acid
A11 is a charged amino acid
A12 is a charged or polar or hydrophobic amino acid
A13 is a hydrophobic or charged amino acid or a neutral amino acid
A14 is a hydrophobic or polar amino acid or charged or neutral amino acid
A15 is a charged or polar or hydrophobic amino acid
A16 is a polar or hydrophobic or charged amino acid or a neutral amino acid
A17 is a polar or charged amino acid a neutral amino acid
A18 is a polar or charged or hydrophobic amino acid
A19 is a polar amino acid or a neutral amino acid
A20 is a hydrophobic or polar amino acid
A21 is a hydrophobic amino acid
A22 is a polar or hydrophobic amino acid.

2. A nucleotide sequence coding for the amino acid sequence of formula (I) as defined in claim 1.

3. A modified PME wherein the modified PME is obtainable from providing an initial PME that does not comprise an amino acid sequence of the formula (I); and modifying the initial PME so that it does comprise an amino acid sequence of the formula (I) as defined in claim 1.

4. A modified PME wherein the modified PME is obtainable from providing an initial PME that does comprise an amino acid sequence of the formula (I); and modifying the initial PME so that it does not comprise an amino acid sequence of the formula (I) as defined in claim 1.

5. A gene coding for a modified PME wherein the gene coding for the modified PME is obtainable from providing an initial gene coding for a PME that does not comprise a sequence coding for an amino acid sequence of the formula (I); and modifying the initial gene coding for the PME so that it does comprise a nucleotide sequence coding for an amino acid sequence of the formula (I) as defined in claim 1.

6. A gene coding for a modified PME wherein the gene coding for the modified PME is obtainable from providing an initial gene coding for a PME that does comprise a sequence coding for an amino acid sequence of the formula (I); and modifying the initial gene coding for the PME so that it does not comprise a nucleotide sequence coding for an amino acid sequence of the formula (I) as defined in claim 1.

7. A process of modifying a PME comprising the steps of providing an initial PME that does not comprise an amino acid sequence of the formula (I); and modifying the initial PME so that it does comprise an amino acid sequence of the formula (I) as defined in claim 1.

8. A process of modifying a PME comprising the steps of providing an initial PME that does comprise an amino acid sequence of the formula (I); and modifying the initial PME so that it does not comprise an amino acid sequence of the formula (I) as defined in claim 1.

9. A method of preparing a gene coding for a modified PME comprising the steps of providing an initial gene coding for a PME that does not comprise a sequence coding for an amino acid sequence of the formula (I); and modifying the initial gene coding for the PME so that it does comprise a nucleotide sequence coding for an amino acid sequence of the formula (I) as defined in claim 1.

10. A method of preparing a gene coding for a modified PME comprising the steps of providing an initial gene coding for a PME that does comprise a sequence coding for an amino acid sequence of the formula (I); and modifying the initial gene coding for the PME so that it does not comprise a nucleotide sequence coding for an amino acid sequence of the formula (I) as defined in claim 1.

11. Use of an amino acid sequence of formula (I) for affecting PME activity.

12. Use of an amino acid sequence of formula (I) for affecting enzymatic activity.

13. A modified enzyme comprising the amino acid sequence of the formula (I) as defined in claim 1

14. A foodstuff prepared by use of the amino acid sequence of the formula (I) as defined in claim 1

15. A foodstuff according to claim 14 wherein the foodstuff is a pectin.

16. A modified PME comprising the amino acid sequence of formula (I) as defined in claim 1.

17. A process of de-methylating pectin comprising contacting pectin with a modified PME comprising the amino acid sequence of formula (I) as defined in claim 1.

18. A process of preparing a foodstuff comprising using a de-methylated pectin, wherein the de-emtylated pectin is prepared by contacting pectin with a modified PME comprising the amino acid sequence of formula (I) as defined in claim 1.

19. An amino acid sequence substantially as described herein.
